(19) United States
(12) Patent Application Publication
(10) Pub. No.: US 2018/0133046 A1
(43) Pub. Date: May 17, 2018

(54) CUSTOM SPINAL ORTHOSIS, METHODOLOGY AND WEAR COMPLIANCE

(71) Applicant: Osteodirit, Inc., Irvington, NY (US)

(72) Inventor: Michael G. Vitale, Irvington, NY (US)

(73) Assignee: Osteodirit, Inc., Irvington, NY (US)

(21) Appl. No.: 15/354,824

(22) Filed: Nov. 17, 2016

(51) Int. Cl. A61F 5/02; A61B 5/055; A61B 6/00; G06F 19/00; G05B 19/042

(52) U.S. Cl. CPC A61F 5/02; A61B 5/055; A61B 6/505; G05B 2219/2652; G06F 19/3481; G05B 19/042; G06F 19/3406

(57) ABSTRACT

A system, device and methodology for treatment of spinal deformities is provided. Characteristics associated with a patient with a spinal deformity are analyzed and recorded. The characteristics include sagittal balance which is a measure of balance of the spine in a sagittal plane. A clinician generates a prescription outlining a desired correction based on the characteristics for treatment of the spinal deformity. A custom orthosis is manufactured in view of the desired correction provided by the prescription. The custom orthosis provides for monitoring of patient's wear characteristics and compliance with prescribed orthosis wear times.

### TECHNICAL FIELD

This disclosure is related generally to the treatment of scoliosis, and more particularly, to a system, device and methodology for treating a scoliosis patient with a custom spinal orthosis created in view of input of patient characteristics, the orthosis incorporating sensors to monitor patient's wear characteristics and compliance with prescribed wear times.

### BACKGROUND OF THE INVENTION

As part of a treatment regimen for scoliosis, a physician may prescribe a spinal brace, or orthosis, to a patient to aid in correcting the deformity of the patient's spine. Patients may be prescribed a pre-fabricated external spinal orthosis that, by definition, is not customized to meet the patient's precise specifications and needs. For example, a patient may be prescribed a spinal orthosis that does not take into account certain clinical characteristics that are unique to the patient. Furthermore, pre-fabricated spinal orthoses may not fit properly relative to a patient's height or weight. Consequently, prefabricated orthoses may not function optimally, or effectively, to address the patient's spinal condition.

Therefore, what is desirable is a custom-fabricated orthosis designed with physician input to address certain individualized patient characteristics and correction parameters. What is further desirable is for such an orthosis to include a method and device for monitoring its effectiveness on the patient, as well as the patient's compliance with wearing it properly, including for the prescribed time periods.

### SUMMARY

A system, device and methodology for treatment of spinal deformities is provided. In an implementation, a scan of a body is performed. One or more of multiple characteristics associated with the body are determined in view of the scan. In an implementation, a clinician may determine the characteristics. In another implementation, a computing device may determine the characteristics. The characteristics include sagittal balance. A clinician or computing device may determine a desired correction of the body in view of the sagittal balance. The desired correction is then provided to a manufacturing device to generate an orthosis. The orthosis includes a housing and a strap that is generated in view of the desired correction. Compliance and quality of wear of the orthosis may be monitored using an orthosis monitoring device.

In another implementation, a scan of a body is accessed to analyze a spinal deformity. A value for pelvic incidence, a characteristic unique to each patient, is determined from the scan. The clinician determines a desired correction to treat the body in view of the pelvic incidence. The desired correction is then provided to a manufacturing device to generate an orthosis.

In another implementation, a database may store an input of sagittal balance that is associated with a scoliotic spine of a patient. The input of the sagittal balance includes an input of a pelvic incidence. A device stores a prescription for desired correction of the scoliotic spine determined in view of the sagittal balance. A custom orthosis is manufactured by a manufacturing device in view of the desired correction.

### BRIEF DESCRIPTION OF DRAWINGS

The disclosure may be understood more fully from the detailed description given below and from the accompanying drawings of various implementations of the disclosure. The drawings, however, should not be taken to limit the disclosure to the specific implementations, but are for explanation and understanding only.

FIG. 1 is a block diagram of an exemplary custom spinal orthosis system for creating a custom orthosis and monitoring related to the orthosis;

FIG. 2A illustrates a lateral view of a human spine;

FIG. 2B illustrates a posterior view of a scoliotic spine;

FIG. 3A illustrates a posterior view of a person's torso exhibiting a normally shaped spine;

FIG. 3B illustrates a posterior view of a person's torso exhibiting deformed spinal curvature;

FIG. 3C illustrates various spinal alignments relative to traditional orthoses;

FIG. 4 illustrates a scoliotic spine;

FIG. 5 illustrates an exemplary custom orthosis;

FIG. 6A illustrates exemplary sacral and pelvic bones and associated parameters;

FIG. 6B illustrates exemplary pelvic parameters;

FIG. 6C illustrates yet other exemplary pelvic parameters;

FIG. 7 is a flow chart illustrating a method for prescribing a customized orthosis based on sagittal balance, according to an implementation of the disclosure;

FIG. 8 illustrates an exemplary custom orthosis and an orthosis monitoring device, according to an implementation of the disclosure;

FIG. 9 is a flow chart illustrating a method of implementing an orthosis monitoring device, according to an implementation of the disclosure;

FIG. 10 is a flow chart illustrating a method for comparing factors at an orthosis monitoring device, according to an implementation of the disclosure;

FIG. 11 is a flow chart illustrating a method for monitoring patient characteristics to determine a change, according to an implementation of the disclosure;

FIG. 12 is a flowchart illustrating a method for prescribing a customized orthosis based on assessment of a scan of a spine, according to an implementation of the disclosure;

FIG. 13 is a flowchart illustrating a method for comparing factors for providing a message or alert, according to an implementation of the disclosure;

FIG. 14 is a flowchart illustrating a method for providing a display at a user device, according to an implementation of the disclosure; and

FIG. 15 illustrates a block diagram of an illustrative computing device operating in accordance with the examples of the present disclosure.

### DETAILED DESCRIPTION

A technician, physician, nurse, etc. (herein referred to collectively or individually as medical personnel or clinician) may examine a patient with spinal deformities in order determine an appropriate treatment and provide an improved state of spinal balance. The clinician may perform a full or targeted scan of the patient's body. Using the body scan, the clinician may determine one or more patient characteristics unique to a patient's body.

Spinal balance is an important factor to allow for upright posture and activity with minimal energy expenditure. A deformed spine may be out of balance in a frontal and/or sagittal plane in a variety of pathological conditions. (A frontal plane is viewed from the back of the body, also referred to as a coronal plane. A sagittal plane is a vertical plane that runs up and down a person's body.) Continued focus and research by the medical community has shed much light, recently, on the complex relationships between spinal balance and curve progression (which is a progression of a spinal curve beyond a normal curvature) in adolescent idiopathic scoliosis (AIS).

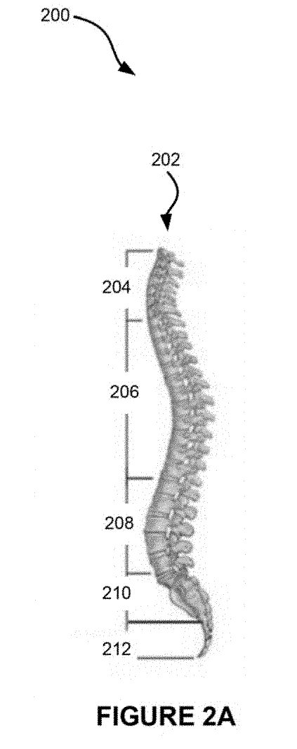

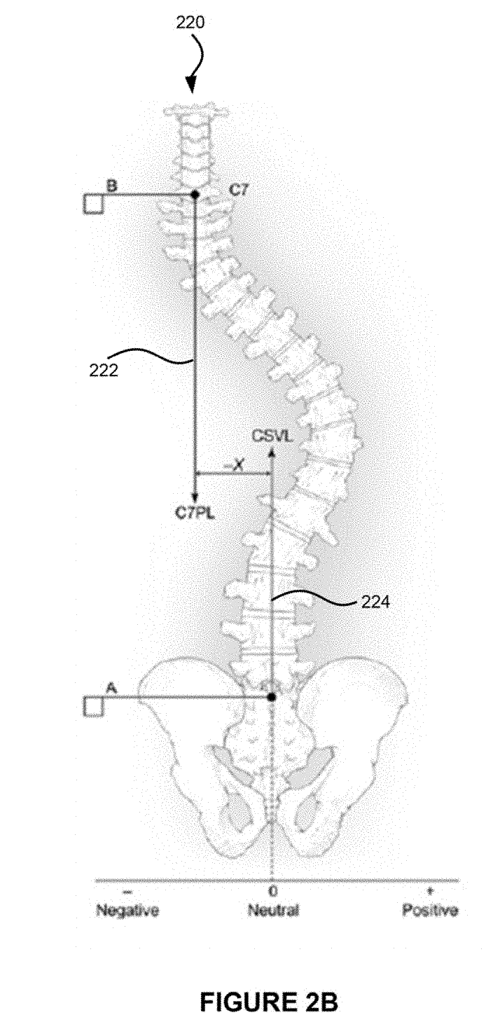

Frontal balance is considered a less complex assessment. It is assessed as the horizontal distance between a central sacral vertical line (CSVL) and a C7 plumb line (C7PL). The CSVL is a vertical line drawn through the middle of the S1 vertebra. The C7PL is a vertical line drawn through the center of the C7 vertebra. By convention, frontal balance is thought to be positive (+X) if the CSVL falls to the left of the C7PL, and negative (−X) if the CSVL falls to the right of the C7PL. FIG. 2B illustrates a posterior view of a scoliotic spine 220, wherein line 222 is the C7PL, line 224 is the CSVL, and the distance between them is −X.

Traditionally, spinal bracing has focused on maximizing correction of the primary curve rather than improving overall frontal spinal balance. Sagittal balance in AIS, as compared to frontal balance, is more complicated and less well-understood. By convention, sagittal balance (also called SVA or spinal vertical axis) is defined as positive when the C7PL is anterior to the line drawn vertically from the posterior superior corner of the sacrum (which is a triangular bone at the bottom of the spine). Historical simplistic definitions may look at sagittal balance as a relationship between thoracic kyphosis (curvature of the rib cage portion of the spine) and lumbar lordosis (reverse curvature or sway of the lumbar portion of the spine). Traditional concepts of spinal orthosis design may have typically resulted in a loss of lumbar lordosis with subsequent development of positive sagittal balance.

More recently, it has become understood that sagittal balance may be primarily driven by the relationship of compensatory thoracic kyphosis, lumbar lordosis and the fixed pelvic parameter of pelvic incidence (PI). Pelvic incidence, or PI, is defined as an angle subtended by a line drawn perpendicular from the sacral endplate at its midpoint, and the line drawn from this midpoint to the midpoint of the femoral heads axis. Details regarding PI are described herein.

While the PI is fixed after adolescence, the PI generally increases as with development of the child reflected in horizontalization of the pelvis and resulting in an increase in lumbar lordosis. Evidence exists suggesting that AIS results from and progresses as a result of imbalance between PI and spinal alignment. Moreover, traditional spinal bracing did not take into account the wide variability in PI and the effect of bracing on spino-pelvic imbalance.

As part of a treatment regimen, the clinician may determine an amount of correction to be performed on the patient's body. The clinician may prescribe a customized orthosis that is based on one or more characteristics, such as sagittal balance, to determine how much correction to perform on a patient's spine to alleviate a spinal deformity. Since the specific values of sagittal balance are unique to each patient, prescribing a custom-fabricated orthosis that takes those parameters into account may more effectively correct a spinal deformity.

The desired correction and prescription, as determined by the clinician, may be provided to a manufacturing device to generate a custom-fabricated orthosis. The orthosis may include a housing and one or more straps that are manufactured according to the specifications and needs of the patient, and specifically, based on the one or more patient characteristics and a desired correction as determined by a clinician.

Previous solutions for providing pre-fabricated orthoses do not account for unique patient characteristics such as sagittal balance. Further, patients that wear pre-fabricated, non-customized orthoses may experience unnecessary discomfort, which may discourage them from wearing the orthosis for the prescribed amount of time. Moreover, these patients may wear the orthosis incorrectly, which may hinder the treatment for their spinal deformity.

What is needed is an orthosis that utilizes the concepts of pelvic incidence, sagittal balance, frontal balance and other factors as a philosophical underpinning and clinical foundation towards design. Brace design and fabrication may be focused around achieving frontal and sagittal balance which is customized to each individual's unique anatomy. Moreover, the orthosis may have the ability to continually optimize balance both through differential tightening of straps, through the addition of interchangeable pads, and with use of biofeedback garnered from an orthosis monitoring device.

Implementations of the present disclosure address the shortcomings of the previous solutions by utilizing patient characteristics including sagittal balance in order to determine a precise correction scheme and generate an orthosis based on the desired correction scheme.

Various aspects of the above referenced methods and systems are described in details hereinbelow by way of examples, rather than by way of limitations.

A person's spine includes thirty-three individual bones called vertebrae that are stacked one on top of the other. The spine (also called a spinal column) functions as the main support for a person's body and enables a person to move, bend, twist, etc. Details regarding the spine are described herein with respect to FIG. 2A.

FIG. 2A illustrates a lateral view 200 of a human spine 202. Spine 202 is divided into multiple segments. These segments are called the cervical spine 204, thoracic spine 206, lumbar spine 208, sacrum 210 and coccyx 212.

The cervical spine is also referred to as the neck. The cervical spine supports the weight of a person's head (weighing approximately ten pounds). There are seven vertebrae within the cervical spine. These vertebrae are individually referred to as C1 through C7. The cervical spine has the greatest range of motion of all the spinal segments due to the unique nature of vertebra C1 and vertebra C2.

Vertebra C1, also referred to as the atlas, is ring-shaped, and the only cervical vertebra to not have a vertebral body. C1 connects to the base of the skull, known as the occiput. This connection is known as the atlanto-occipital joint. The atlanto-occipital joint allows for a nodding up and down, or forward and backward motion of a head, and is responsible for about 50% of the head's forward and backward range of motion.

Vertebra C2, called the axis, has a large bony protrusion, known as the odontoid process, that points up from its vertebral body, and fits into the ring-shaped atlas above it. The C1-C2 joint is known as the atlanto-axial joint and enables the atlas to rotate about the axis. About 50% of the head's rotation occurs at this joint.

The thoracic spine is also referred to as the mid back. A person's thoracic spine is connected to his/her rib cage. There are twelve vertebrae within the thoracic spine. These vertebrae are individually referred to as T1 through T12. The thoracic spine has a limited range of motion.

The lumbar spine is also referred to as the low back. The lumber spine bears the weight of a person's body. There are five vertebrae within the lumber spine. These vertebrae are individually referred to as L1 through L5. Vertebrae L1 to L5 are larger in size in comparison to the other vertebrae of the spine, which also allows these vertebrae to absorb stress placed on the body caused by carrying or lifting heavy objects, for example.

The sacrum is connected to a person's hip bone or ilium. There are five vertebrae within the sacrum. These vertebrae are individually referred to as S1 through S5. The five vertebrae, which are fused to one another, when combined together with the ilium form a pelvic girdle that is ring shaped.

The coccyx is also referred to as the tailbone. The coccyx is made up of four fused vertebrae that attach to ligaments and muscles of a pelvic floor (not depicted).

The pelvis is formed by the right and left hip bones (not depicted) along with the sacrum and the coccyx.

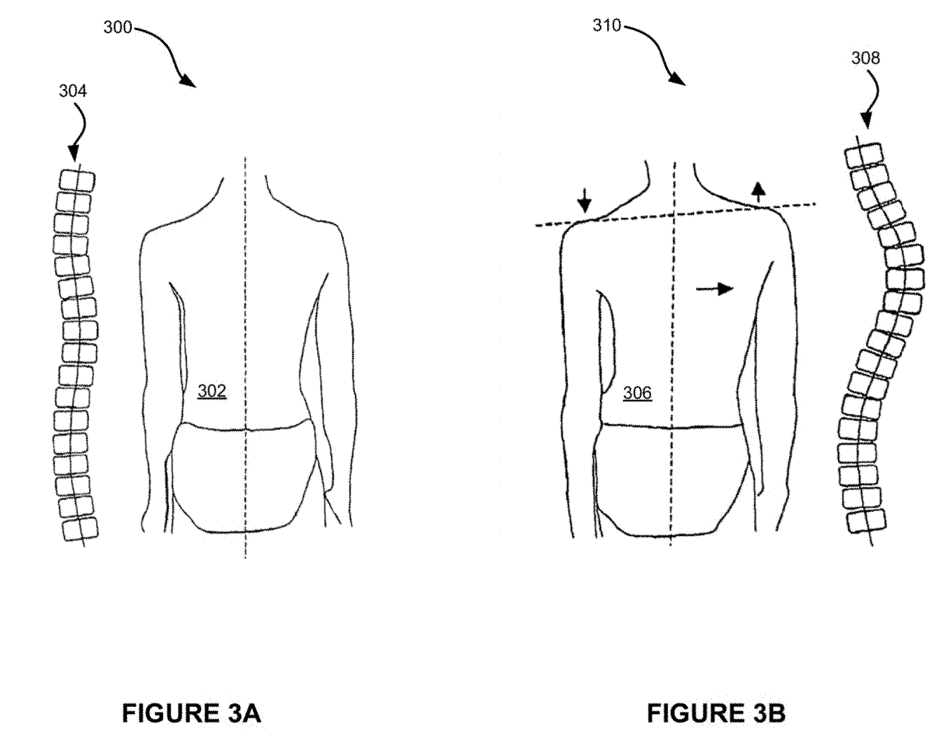

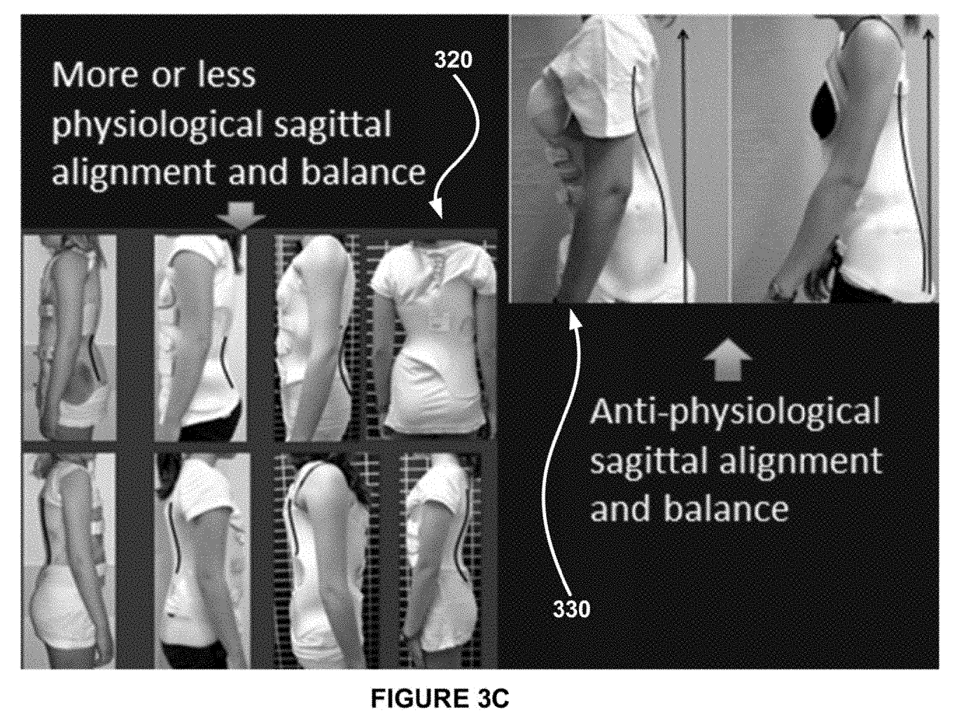

As shown in FIG. 2A, cervical spine 204, thoracic spine 206 and lumbar spine 208 are each curved. Spine 202, as depicted in FIG. 2A, is a normally shaped spine. Abnormalities in the spine, however, may exist in some people. In particular, a lumber spine that is abnormally curved is referred to as exhibiting excessive lordosis, hyperlordosis, or sway back. A thoracic spine that is abnormally curved is referred to as exhibiting excessive kyphosis, hyperkyphosis, or hunchback. A deformed spine in the coronal plane that is abnormally curved is known as scoliotic. Details regarding abnormalities in the spine are described herein with respect to FIG. 3B.

A portion of the spine houses the spinal cord (not depicted), which is approximately eighteen inches long. The spinal cord is part of the central nervous system and transmits and receives messages to and from the brain and body. The spinal cord is made up of thirty-one pairs of spinal nerves that branch off the spinal cord. Damage to the spinal cord may be temporary or permanent and result in a loss of sensor and/or motor functions. For example, damage to the spinal cord within the thoracic or lumber spine area may cause leg or trunk motor and sensory loss (referred to as paraplegia). Damage to the spinal cord within the cervical spine area may cause arm or leg motor loss (called tetraplegia).

FIG. 3A and FIG. 3B exhibit a normally shaped spine and a deformed spinal curvature, respectively. FIG. 3A illustrates a posterior view 300 of a person's torso 302 exhibiting a normally shaped spine 304.

A normally shaped spine has a natural “S”-shaped curve when viewed from the side or along a sagittal (also referred to as anteroposterior) plane. A sagittal plane is a vertical plane that runs up and down a body to divide it into left and right sections. A normally-shaped spine has a vertically straight shape when viewed from the back of the body or along a coronal (also referred to as frontal) plane. A coronal plane divides a body into dorsal and ventral (back and front, or posterior and anterior) portions.

As described above, normal sagittal balance refers to normal balance or harmony of a spine in the sagittal plane. Normally shaped spine 304 in FIG. 3A may be associated with normal sagittal balance. Sagittal imbalance refers to a front-to-back (in the sagittal plane) imbalance of the spine (see FIG. 3B). Thus, sagittal balance is the natural and proper spinal curvature of the spine. Sagittal balance may depend on several factors. One of these factors includes pelvic incidence (described herein below).

The term lordosis refers to the normal inwardly-shaped spinal curvature of the cervical and of the lumbar spine in the sagittal plane. The term kyphosis refers to the normal outwardly-shaped spinal curvature of the thoracic spine and of the sacrum in the sagittal plane.

FIG. 3B, on the other hand, illustrates a posterior view 310 of a person's torso 306 exhibiting a deformed spinal curvature. In FIG. 3B, the person has a deformation of the back. As depicted in FIG. 3B, the deformedly shaped spine 308 has an abnormal curvature when compared with spine 304 in FIG. 3A. Torso 306 in FIG. 3B may be improved over time by use of an orthosis so that it can be reshaped and appear closer to torso 302 in FIG. 3A.

One type of deformed spinal curvature of the lumbar spine in the sagittal plane is known as excessive lordosis. Excessive lordosis is a skeletal deformity where the inward lordotic curve is exaggerated. Excessive lordosis has also been known to occur in the cervical spine. A person exhibiting excessive lumbar and cervical lordosis may appears to have an exaggerated posture whereby the buttocks and chest thrust outwardly. Symptoms of such a condition may include back pain, as well as restricted movement and difficulty in movement.

Excessive kyphosis is an abnormally convex curvature typically of the thoracic spine. Excessive kyphosis may cause a hunch or hump, also known as roundback or Kelso's hunchback. A spine exhibiting normal kyphosis has a spinal curvature that is typically between twenty and forty-five degrees when measured from the first cervical vertebra (C1) over twelve consecutive vertebrae to the fifth thoracic vertebra (T5). A spine exhibiting excessive kyphosis has a spinal curvature that is typically more than forty-five degrees. A person that has a severe case of excessive kyphosis may experience cardiovascular and neurological conditions, digestion issues, difficulty breathing and severe pain.

A deformed spine in the coronal plane having a spinal curvature of ten degrees or more is known as scoliosis. Scoliosis may be associated with an axial twisting of some of the vertebrae. The spinal curvature of an individual with scoliosis may appear to be “C” or “S”-shaped in the frontal plane. While most cases of scoliosis do not cause pain, severe cases of scoliosis may create difficulty in breathing and may cause significant pain.

There are several types of scoliosis. Congenital scoliosis is caused by an abnormality of the spine at birth. Neuromuscular scoliosis is caused by abnormal muscles or nerves. Individuals that have spina bifida, cerebral palsy, or are paralyzed often have neuromuscular scoliosis. Degenerative scoliosis may result from a traumatic injury or illness, bone collapse, previously performed major back surgery, or osteoporosis (which is a thinning of the bones). Idiopathic scoliosis is the most common type of scoliosis. Idiopathic scoliosis has no specific cause, however, it may be hereditary.

Scoliosis may initially develop in the cranium, or skull, and the distortion in position of cranial bones may cause the spine to become twisted and side-bent. This may further lead to the pelvis becoming side-tilted and twisted, the positions of the ribs becoming asymmetrical, and so forth. If scoliosis is left untreated, additional twisting and bending of the spine may continue over time. Idiopathic scoliosis in children is known as adolescent idiopathic scoliosis (AIS).

Referring again to FIG. 3A, when a normally shaped spine 304 is viewed from the side or along a sagittal plane, it has a natural “S”-shaped curve. When viewed along a coronal plane, a normally shaped spine 304 appears to be vertically straight. Referring again to FIG. 3B, when a deformedly shaped spine 308 is viewed along a coronal plane, it appears to be curved. FIG. 3B depicts a form of scoliosis. Deformedly shaped spine 308 causes an obliqueness of the shoulders, which is shown by the horizontal dashed line. As depicted, the left shoulder is lower than the right shoulder, which causes an asymmetry in the torso. The vertical dashed line in FIG. 3B represents an ideal mean position of the back of the torso. The torso in FIG. 3B is not symmetrical along the ideal mean position. Unlike the torso in FIG. 3B, the vertical dashed line in FIG. 3A, which represents an ideal mean position of the back of the torso, divides the torso into two symmetrical portions.

A clinician may initially diagnose scoliosis through visual observation. Scoliosis in a child may be detected during a screening examination performed at school, for example. A child with scoliosis may have uneven shoulders, a prominent shoulder blade, uneven waist, or lean to one side of the body. Scoliosis, and the type of scoliosis, are determined by a bone examination and an x-ray to determine the angles of the spinal curve.

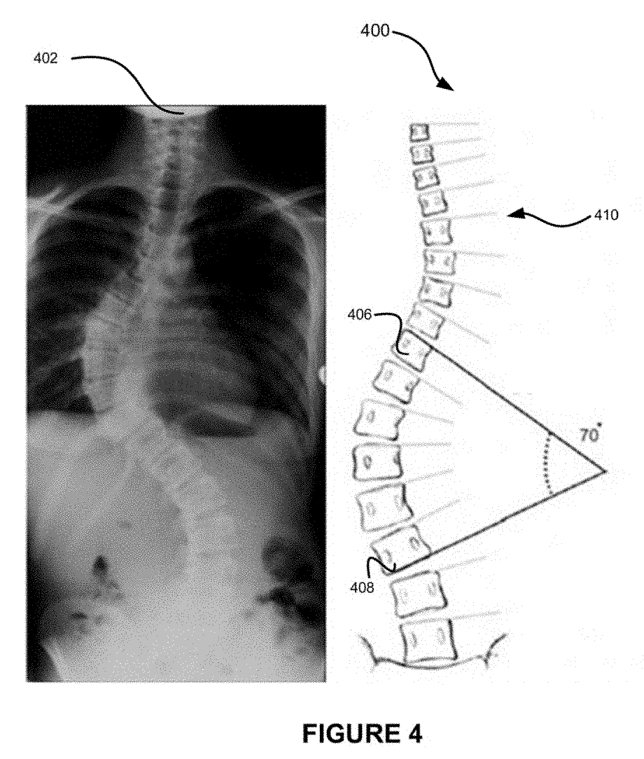

There are various treatments for scoliosis. In order to determine a course of treatment, clinicians may quantify the magnitude of the spinal deformity. One way to quantify the magnitude is by using a measurement called the Cobb angle. To measure a Cobb angle, the clinician determines which two vertebrae are the end vertebrae of a curve deformity. Thus, the clinician identifies the vertebrae at the upper and lower limits of the curve deformity. The vertebra at the upper limit of the curve deformity is referred to as a superior end vertebra. The vertebra at the lower limit of the curve deformity is referred to as an inferior end vertebra. The Cobb angle of the curve deformity is formed by an intersection of the following two lines: one line that is parallel to an endplate of the superior end vertebra, and another line that is parallel to an endplate of the inferior end vertebra. The Cobb angle may be analyzed to diagnose scoliosis. Specifically, scoliosis occurs when a lateral spinal curvature has a Cobb angle of ten degrees or greater. An exemplary calculation of the Cobb angle is described herein with respect to FIG. 4.

FIG. 4 illustrates a scoliotic spine 400. An x-ray 402 depicts a curve deformity. A rendering 410 of the spine imaged in x-ray 402 shows a superior end vertebra 406 and an inferior end vertebra 408. The Cobb angle of the curve deformity is formed by an intersection of a line that is parallel to an endplate of superior end vertebra 406, and a line that is parallel to an endplate of inferior end vertebra 408. As described above, lateral spinal curvature having a Cobb angle of ten degrees or more is indicative of scoliosis. In the depicted implementation, the Cobb angle is seventy degrees, which is above ten degrees, and therefore, indicative of scoliosis.

In severe cases of scoliosis (Cobb angle of above forty degrees) where spinal curves extend beyond forty to fifty degrees, surgery may be performed. Surgery involves inserting metal implants, including spinal screws and rods, inside the body to correct some of the curvature. During surgery, the metal implants aid in holding the spine in a correct position while one or more bone grafts are inserted. Then, this construct leads to a rigid fusion of the bones in the area of the curvature. When two or more vertebrae are caused to fuse together, this type of procedure is referred to as a spinal fusion.

Younger children may not undergo spinal fusion surgery as their bones are growing and the fusion may stunt the growth of the fused part of the spine. Other surgical techniques, as alternatives to spinal fusion, may be used to address spinal deformities in younger children.

Whether or not spinal surgery is performed, treatment for scoliosis involves use of an orthosis. Orthoses may typically be used to treat deformed spinal curves that range in Cobb angle from twenty-five to forty degrees. Younger children who have yet to finish growing and have immature skeletons may also benefit from orthoses. Treatment of skeletal deformity, however, may be difficult as it involves extensive recovery and/or orthosis use time. Orthosis treatment for scoliosis typically involves use of an orthosis for at least twelve hours per day in some instances, and up to twenty to twenty-three hours per day.

Treatment of idiopathic scoliosis using an orthosis may ideally stop the progression of a spinal curve, but may not reduce the degree of the curve or the amount of angulation that is already present. Therefore, early detection may be helpful in the treatment of scoliosis.

Existing solutions do not offer unique orthoses that account for a patient's particular body type, spinal configuration, patient characteristics and other factors. Existing solutions also do not monitor use of the orthosis as it relates to its effectiveness on a patient. Rather, existing solutions offer limited sizes and pre-fabricated orthoses, which are not unique to a patient's body and needs. For example, a clinician may measure a person's height, weight and curve of the spine. The clinician may then choose a pre-fabricated orthosis from a limited selection of pre-fabricated orthoses in view of the measured physical aspects. The clinician may choose a smaller size orthosis for a younger patient having an immature skeleton and a larger size orthosis for an adult. Accordingly, existing orthoses do not account for various characteristics of a patient's spine and pelvis, which may be unique to his/her condition. Specifically, existing orthoses do not account for sagittal balance or other unique characteristics of a patient's pelvis and spine. By contrast, a custom orthosis based on these patient characteristics is desirable as such a custom orthosis would ensure a proper fit, and a user would be more likely to derive a greater benefit from the orthosis, be more comfortable when wearing the orthosis, and, in turn, continue using the orthosis.

Some studies show that progression of the AIS curve after traditional bracing may be highly dependent on spinopelvic balance (or the balance of the spine and pelvis). The results of these studies of patients using traditional orthoses are as follows:

Progression at 6 months post bracing

| | |
|---|---|
| a. SVA >4 cm | 9% |
| b. SVA <4 cm | 3% |
| c. Frontal Imbalance >6 cm | 11% |
| d. Frontal Imbalance >2 cm | 4% |

FIG. 3C illustrates various spinal alignments resulting from wear of traditional orthoses. The configurations shown in section 320 may be a result of more or less physiological sagittal alignment and balance. The configurations shown in section 330 may be a result of anti-physiological sagittal alignment and balance. In section 330, the patients depicted may have been treated without accounting for physiological patient characteristics.

Existing orthosis solutions may also not monitor effectiveness of an orthosis on the patient as well as a patient's compliance with properly wearing of the orthosis. Wear compliance refers to the ability to use the orthosis for a prescribed period of time. In one example, a patient who is prescribed a pre-fabricated and non-customized existing orthoses may experience discomfort in wearing the orthosis for an extended period of time. In another example, a patient may not accurately track the time he/she uses the orthosis. In yet another example, a patient may not feel motivated to wear the orthosis for the prescribed time. In yet another example, a patient may undergo a change in his/her body weight, height, sagittal balance, pelvic incidence, etc. which may lead to the orthosis causing the patient increased discomfort.

Moreover, a patient may be compliant with respect to the wear-time of an orthosis but still experience issues with respect to the quality of orthosis use. Quality of orthosis use is independent from wear-time compliance and refers to a measure of how well an orthosis is worn for its intended purpose. Quality of orthosis use may measure if the orthosis (e.g., a brace) is properly tightened, properly placed, etc. Suppose that a patient wears an orthosis for the prescribed twenty hours a day, however, the straps of the orthosis are too loose. The patient may meet wear-time compliance of the orthosis but may not meet the quality of orthosis wear.

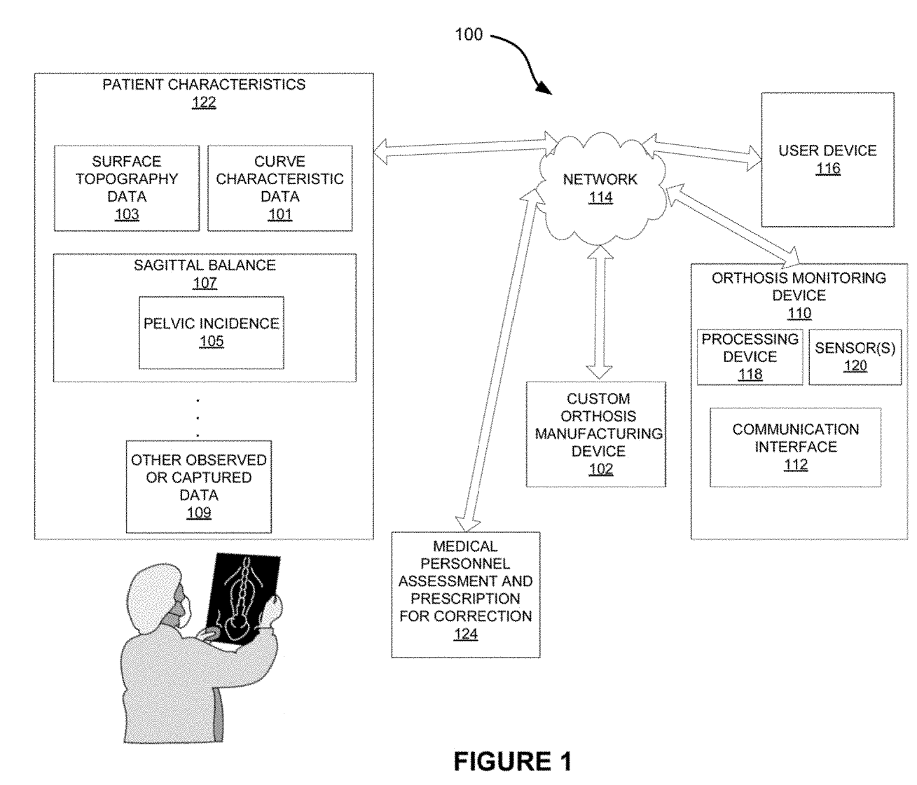

Thus, existing orthotic solutions are limited and do not offer custom solutions based on a patient's characteristics such as sagittal balance. Existing solutions also do not monitor the effectiveness of an orthosis on a patient, as well as the patient's quality of use and compliance with wearing the orthosis for a prescribed time period. A custom spinal orthosis system that addresses the problems in existing solutions is described herein below with respect to FIG. 1.

FIG. 1 is a block diagram of an exemplary custom spinal orthosis system 100 for creating a custom orthosis and monitoring related to the orthosis. System 100 includes, patient characteristics 122, a medical personnel assessment and prescription for correction 124, a custom orthosis manufacturing device 102, an orthosis monitoring device 110, a user device 116, and a network 114.

Patient characteristics 122 may include curve characteristic data 101, surface topography data 103, pelvic incidence 105, sagittal balance 107, and other observed or captured data 109. Patient characteristics 122 may include additional characteristics data than shown in FIG. 1. In an implementation, a clinician may determine patient characteristics by observing a scan or an image of a patient's body. In another implementation, a computing device may determine patient characteristics. In an implementation, patient characteristic 122 may be a database or a type of storage facility. In another implementation, patient characteristic 122 may be a part of a computing device. In yet another implementation (not depicted), patient characteristics 122 may be stored in separate locations, databases, etc.

Orthosis monitoring device 110 includes a processing device 118, one or more sensors 120, and a communication interface 112. Details regarding orthosis monitoring device 110 are described herein.

In the depicted implementation, each or some of medical personnel assessment and prescription for correction 124, patient characteristics 122, custom orthosis manufacturing device 102, orthosis monitoring device 110 or user device 116 of system 100 may be connected to one another via network 114. In another implementation, each or some of medical personnel assessment and prescription for correction 124, patient characteristics 122, custom orthosis manufacturing device 102, orthosis monitoring device 110 or user device 116 of system 100 may communicate directly with one another via a direct connection (e.g., a bus, etc.).

In one implementation, network 114 may include a public network (e.g., the Internet), a private network (e.g., a local area network (LAN) or wide area network (WAN)), a wired network (e.g., Ethernet network), a wireless network (e.g., an 802.11 network or a Wi-Fi network), a cellular network (e.g., a Long Term Evolution (LTE) network), routers, hubs, switches, server computers, and/or a combination thereof.

Although a single user device is depicted, in other implementations, multiple user devices may be included in system 100. The user devices may include one or more computing devices such as personal computers (PCs), laptops, mobile phones, smart phones, tablet computers, netbook computers etc. User device 116 may include a display device that displays graphical and textual data. User device 116 may include one or more applications (apps) that allow viewing of data related to orthosis wear.

If a patient with a spinal curve wishes to be assessed by a clinician to receive treatment, as depicted in FIG. 1, a clinician may order, complete, or otherwise acquire a scan of a patient's body (and including the patient's spine and pelvis). Patient characteristics 122 may be extracted from one or more scans, imaging, x-rays, EOS solutions (the description of which may be found at www.eos-imaging.com, and is incorporated by reference herein; collectively referred to herein as “EOS”) and/or other means (hereinafter referred collectively or individually as scans). The clinician may assess patient characteristics 122 to create medical personnel assessment and prescription for correction 124. In an implementation, for treatment of a scoliotic spine, medical personnel assessment and prescription for correction 124 may include a desired correction of the scoliotic spine determined in view of a patient characteristic (e.g., sagittal balance). In an implementation, the desired correction may be a correction of a frontal balance of the body within a front balance correction range. In another implementation, the desired correction may be a correction of the sagittal balance within a sagittal balance correction range. The range may be measured in increments of millimeters. For example, the range may be specified within ten millimeters (or one centimeter).

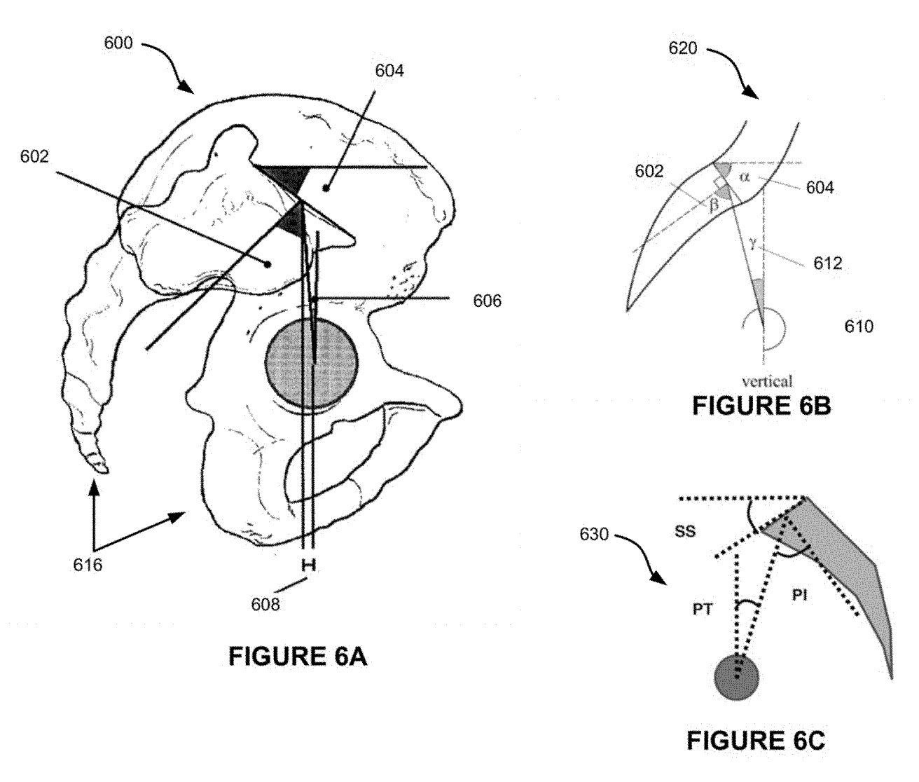

Patient characteristics 122 include curve characteristic data 101, surface topography data 103, sagittal balance 107, which includes pelvic incidence 105, and/or other observed or captured data 109. In an implementation, the medical personnel may input various characteristics into patient characteristics 122. In another implementation, the various characteristics may be determined by a computing device and either input by the computing device or by the medical personnel into patient characteristics 122. Details regarding determination of patient characteristics such as pelvic incidence and sagittal balance are described hereinbelow with respect to FIGS. 6A, 6B and 6C.

Body scans may be performed on a portion of a person's body (e.g., the posterior of the torso to capture the spinal column) or on the entire body. Imaging such as magnetic resonance imaging (MRI) may be used to capture details of the spine and/or other body parts. X-rays may capture images of the spine or other bones. EOS may be used to capture a portion of or the entire skeletal structure of a patient in 3D. As opposed to an x-ray, which captures images in 2D, EOS produces three-dimensional modeling of a skeletal structure from every angle (360 degrees).

Based on the characteristics of the patient, the clinician may determine a course of action to take in order to correct the patient's spinal curve. The clinician may conclude that a treatment, such as an orthosis, is to be administered. As such, the clinician may write a prescription for correction for the patient. In an implementation, the clinician may input additional patient characteristics information or updated patient characteristics information into patient characteristics 122.

FIG. 6A illustrates exemplary sacral and pelvic bones 600 and associated pelvic parameters, according to an implementation of the disclosure. The pelvic parameters include a pelvic incidence 602, a sacral slope 604, pelvic tilting 606, and an overhang of vertebra S1 608. Pelvic incidence 602 is defined as the angle between the line perpendicular to the sacral plate (which is the vertebra, as shown) at its midpoint, and the line connecting this point to the middle axis of the femoral heads. A femoral head is the head of the femur, or the highest part of the femur. A pelvic incidence is an anatomical characteristic, which is unique to each individual and is independent of the spatial orientation of the pelvis. The anatomical components involved in the make-up of pelvic incidence are the first three sacral vertebrae, the sacro-iliac joint and the posterior, or rear, segment of the iliac bone. The sacro-iliac joint connects the sacrum with the iliac or pelvic bone on each side of the lower spine.

Pelvic incidence may be considered a constant or fixed parameter because it is an anatomical element and independent of the position of the pelvis, because the mobility of the sacro-iliac joint may be considered negligible, and because it is independent of age once growth is complete.

Sacral slope 604 is defined as the angle between the superior plate 616 of S1 and a horizontal line.

Pelvic tilt 606 is defined as the angle between the line connecting the midpoint of the sacral plate to the femoral heads axis and a vertical line.

Overhang of vertebra S1 608 (with regard to the femoral heads axis) is defined as the distance between the bicoxofemoral axis and the projection to this level of the midpoint of the sacral plate. The bicoxofemoral axis is a midpoint of a line drawn between both femoral heads. Overhang of vertebra S1 608 may be expressed in millimeters. A point posterior to the bicoxofemoral axis is considered positive whereas a point anterior to this axis is considered negative.

The pelvic parameters of sacral slope 604, pelvic tilting 606 and overhang of vertebra S1 608 reflect the sagittal orientation of the pelvis. Pelvic incidence 602, which is a constant associated with each individual, determines the variable parameters of sacral slope 604, pelvic tilting 606 and overhang of vertebra S1 608. Pelvic incidence 602 is the algebraic sum of sacral slope 604 and pelvic tilt 606. The orientation of the pelvis determines the sagittal position of the sacral plate in relation to the femoral heads axis, adapted for each individual by the pelvic incidence. The higher the value of the pelvic incidence, the higher the value of the adapted overhang of S1.

FIG. 6B illustrates exemplary pelvic parameters 620, according to an implementation of the disclosure. FIG. 6B depicts a pelvic incidence 602 (β) and a sacral slope 604 (α) which are each measured the same as in FIG. 6A. FIG. 6B also depicts a pelvic tilt 612 (γ) and a femoral heads axis 610. Pelvic tilt 612 is defined by (1) a line through the midpoint of the sacral plane and midpoint of the femoral heads axis and (2) the vertical line (which is a dashed line) labeled “vertical”.

FIG. 6C illustrates yet other exemplary pelvic parameters 630. As depicted, pelvic incidence (PI) is the sum of pelvic tilt (PT) and sacral slope (SS).

Pelvic incidence, which is an independent and anatomical parameter, determines pelvic orientation and lordosis in a patient. A low value of pelvic incidence implies low values of pelvic parameters and a flattened lordosis whereas a high value implies a well-tilted pelvic orientation and excessive or pronounced lordosis. Pelvic incidence affects the main axis of sagittal balance of the spine. Pelvic incidence may control spinal curves in accordance with the adaptability of the other parameters (sacral slope and overhang of vertebrae).

A person that has scoliosis has a spinal deformity which causes variable degrees of spinal imbalance in both the frontal and sagittal plane. Moreover, scoliosis causes a mismatch between a person's fixed and static pelvic incidence and appropriate pelvic parameters (pelvic tilt and sacral slope). In an implementation, a clinician may determine pelvic incidence or other pelvic parameters (pelvic tilt and sacral slope) and sagittal balance values by analyzing a patient's body scan. In another implementation, a computing device may determine pelvic parameters, including pelvic incidence and a clinician may use the acquired pelvic parameters in determining a desired correction needed for each patient's body.

A person with a high pelvic incidence may be treated with a high amount of reciprocal lumbar lordosis and thoracic kyphosis achieved by an appropriately calibrated and constructed orthosis. In a patient with scoliosis who has a high pelvic incidence, loss of thoracic kyphosis results in spinal imbalance, or sagittal imbalance.

In an implementation, lumbar lordosis may be determined by analyzing the relationship between the pelvic incidence and sacral slope. If a Cobb angle is taken into account, apical (or apex vertebra of the curve deformity) vertebral rotation may provide a three-dimensional aspect to a relationship between the pelvis and spine. An equation of lordosis may be determined based on the Cobb angle and the relationship between the pelvis and spine. In an implementation, a clinician can determine the Cobb angle by analyzing a patient's body scan. In another implementation, the Cobb angle can be determined by a computing device and provided to a clinician.

The three-dimensional (3D) relationship between the pelvis and spine may be important when detecting a curve deformity and prescribing a treatment. In an implementation, the inverse relationship between the Cobb angle and pelvic incidence is as follows. The greater the apical vertebral rotation and the Cobb angle of a curve deformity (i.e., a scoliotic curve), the lower the pelvic incidence and the more restricted the possibilities of achieving economical or desired sagittal balance. Therefore, in scoliosis patients, the curve deformity of lordosis is frequently reduced. The clinician utilizes this relationship to derive a desired correction of each patient's body. The clinician may utilize the pelvic incidence to determine a patient's sagittal balance.

The clinician may recognize that to treat a patient with spinal conditions such as scoliosis, treatment of the spine in both the frontal and the sagittal plane is desired. Thus, the clinician may prescribe orthoses to correct spinal alignment in both the frontal and sagittal planes. Alignment of both the frontal and sagittal planes may balance fixed and anthropometric or uniquely human pelvic parameters to provide optimal and correct posture of a patient when standing upright, referred to as economic standing posture. Alignment of both planes may also improve the patient's ability to move his/her body. Alignment of both the frontal and sagittal planes provides a decrease in energy expenditure during stance and gait as well as a lower chance of curve progression

Referring again to FIG. 1, after the clinician determines a desired correction of a patient's body based on patient characteristics including pelvic incidence and sagittal balance, he/she may create a prescription for correction. The prescription for correction may be provided to custom orthosis manufacturing device 102 to manufacture a custom orthosis that is generated based on the desired correction. The orthosis may include a housing constructed of a hard or rigid material such as a plastic or other material. The orthosis may further include one or more straps that when secured, hold the orthosis in place.

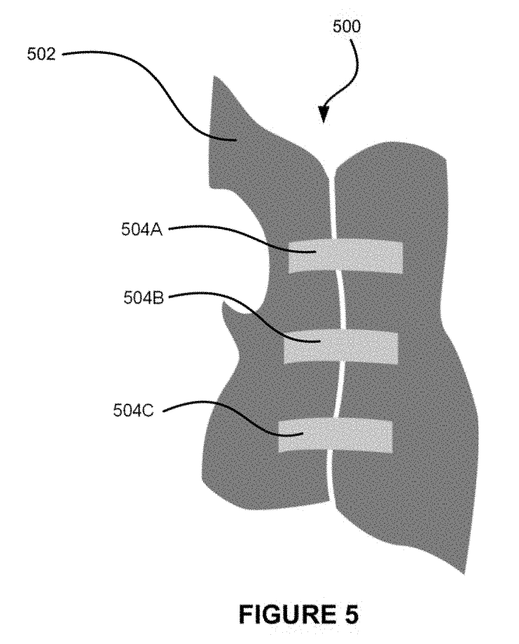

An exemplary custom orthosis 500 is shown in FIG. 5. Custom orthosis 500 includes a housing 502, and straps 504A, 504B, and 504C. Although three straps are shown for exemplary purposes, more or less straps may be included. Although in the depicted implementation, the straps are placed in the front and center of housing 502, in another implementation, one or more of the straps may be placed elsewhere (e.g., on the side or the back of housing 502).

In an implementation, a clinician may prescribe a custom orthosis having a specific shape and size and provide an optimal strap force or pressure based on a patient's characteristics. Optimal strap pressure is a measurement of pressure that the strap exerts on the orthosis in a certain area (e.g., measured in pounds per square inch, etc.). The prescription is used to determine the specifics of the custom orthosis. As described above, the prescription may take into account the sagittal balance which is derived based on pelvic incidence and other patient characteristics. A custom orthosis may be constructed based on a three-dimensional image of a patient's torso (or an area where the orthosis will be worn). The three-dimensional image may be captured and recorded within medical personnel assessment and prescription for correction 124, patient characteristics 122 or elsewhere.

In an implementation, the optimal strap pressure is an exact numerical value. In another implementation, the optimal strap pressure is a range of numerical values. The optimal strap pressure may be set based on the prescription for correction provided as determined by the clinician. In an implementation, the clinician may examine a patient to determine if there is a change in the patient's characteristics. If so, the prescription for correction may be updated in view of the change. In an implementation, if the prescription for correction changes, the optimal strap pressure may also be affected.

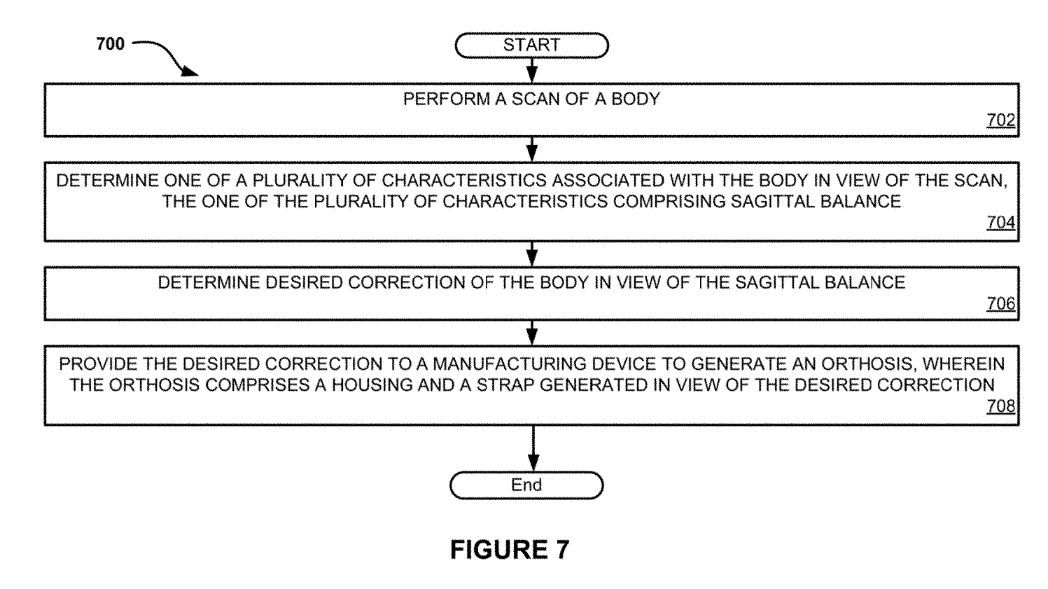

FIG. 7 is a flow chart illustrating a method 700 for prescribing a customized orthosis based on sagittal balance. Method 700, as well as the other methods described herein, may be performed in part or as a whole by a clinician or a computing device. A computing device that may perform method 700 (and other methods) may include processing logic that includes hardware (e.g., circuitry, dedicated logic, programmable logic, microcode, etc.), software (e.g., instructions run on a processing device to perform hardware simulation), or a combination thereof. In describing the method 700, reference is made to FIG. 1 to illustrate an implementation. It is noted that the example provided in FIG. 1 is meant for illustrative purposes, and is not to be considered a limiting implementation.

Method 700 of FIG. 7 begins at step 702 where a scan of a body is performed. Referring to FIG. 1, a clinician scans the body of a patient to assess and prescribe a correction. The medical personnel assessment and prescription for correction 124 may be based on performing a scan of the body which includes imaging the spinal column, other portions of the body, or a full body scan by performing an x-ray, EOS scan and/or a magnetic resonance imaging.

At step 704 of FIG. 7, one or more characteristics associated with the body is determined in view of the scan. The one or more characteristic includes sagittal balance. As shown in FIG. 1, the one or more characteristics are stored and accessible via patient characteristics 122.

In an implementation, the multiple characteristics also includes pelvic incidence. The pelvic incidence is calculated as a sum of the value of the sacral slope and a value of pelvic tilt. The value of the sacral slope and the value of the pelvic tilt are determined by analyzing the scan.

In an implementation, the characteristics may further include curve characteristics data 101 and surface topography data 103 extracted from the x-ray, the EOS scan and/or the magnetic resonance imaging.

At step 706 of FIG. 7, desired correction of the body is determined in view of sagittal balance. The desired correction is included in medical personnel assessment and prescription for correction 124 in FIG. 1.

At step 708 of FIG. 7, the desired correction is provided to a manufacturing device to generate an orthosis. The orthosis includes a housing and a strap generated in view of the desired correction.

Referring again to FIG. 1, medical personnel assessment and prescription for correction 124 is provided to custom orthosis manufacturing device 102 via network 114 or by other means. The custom orthosis includes a housing and a strap that is generated in view of the desired correction for an individual patient. An exemplary custom orthosis is depicted in FIG. 5.

In an implementation, clinician may examine and keep track of a patient's characteristics to determine that a change occurs. The desired correction may be updated to generate an updated desired correction in view of the change. The updated desired correction may then be provided to the manufacturing device to generate an updated orthosis. This may occur, for example, if a patient's characteristics change and he/she may require a new orthosis.

As described above, to help correct a deformity in a patient's spine, the patient may be prescribed a custom-fabricated orthosis that can provide restoration of spinal balance and restoration of a patient's parameters, including pelvic incidence which effects sagittal balance. To construct the custom orthosis, a clinician, a computing device or custom orthosis manufacturing device 102 in FIG. 1 may generate a virtual model of each individual patient. The virtual model may be generated based on merging curve characteristics data of a patient with surface topography data of the patient. The curve characteristic data provides details such as size, shape, curvature, etc., of the patient's spine. The curve characteristic data may be extracted or otherwise obtained from a full-body length three-dimensional scoliosis x-ray of the patient or other means. The curve characteristic data may be measured by a person such as a clinician or by a computing device. The surface topography data used in generation of the virtual model and determination of external body contours of a patient may be extracted or otherwise obtained from a stereophotogrammetry and/or a trunkal body surface scan. A stereophotogrammetry and/or a trunkal body surface scan is used to estimate three-dimensional (3D) coordinates of points on a patient's body by utilizing measurements made in two or more photographic images taken from different positions.

The merged curve characteristics data and surface topography data may be used to define a baseline: the current state of the patient's condition. The merged curve characteristics data and surface topography data may then be input into a computer-aided design (CAD) software program (e.g., ROdin4D CADCAM, etc.) to generate a model of an ideal state of spinal balance. The model of the ideal state of spinal balance provides an image of spinal balance for the patient's spine without abnormalities.

To generate a mold of a custom orthosis for the patient, an impression of the orthosis is created based on the model of the ideal state of spinal balance for the patient and on the baseline current state of the patient's condition. The impression of the base is used to generate the mold of an orthosis.

In an implementation, the custom orthosis may be generated in a way to allow for customization and fine-tuning of spinal balance iteratively over time based on x-rays of the patient wearing the orthosis. In an implementation, a custom orthosis may allow for adjustment of the tension of its straps to allow for customization. In another implementation, removable modular pads may allow for customization of spinal balance. The pads may snap on to and be removed from the orthosis by a button, a snap fastener, a hook and loop fastener or by other means. In yet another implementation, pads may be permanently affixed to the orthosis (e.g., sewn on to the orthosis, glued on to the orthosis, etc.). For example, if the patient has a change in height, weight, or characteristics such as pelvic incidence (which effects sagittal balance), the custom orthosis may be adjusted to account for the changes. In an implementation, the pressure of the strap may need to be adjusted in view of the change. Therefore, the patient may use the same orthosis, which accounts for the changes the patient undergoes.

In an another implementation, in-orthosis examination of the patient and/or scanning of the patient may reveal that the patient needs to obtain a new custom orthosis. As such, a new custom orthosis may be generated and manufactured for the patient taking into account the updated patient characteristics.

A custom orthosis may or may not be form-fitting to a patient's body. In an implementation, a custom orthosis may not form-fit to a patient's body but allow for targeted forces to provide adjustment in specific areas. Although a custom orthosis may be more comfortable for a patient to wear as opposed to a pre-fabricated, non-customized orthosis which may not account for the patient's physical stature, the patient may experience targeted pressure or force. The targeted force exerted by a custom orthosis, which may be felt by the patient wearing the orthosis, aids in the treatment of a scoliotic spine. The targeted forces are applied to target areas which require treatment, and unnecessary forces are avoided. Contrary to a custom orthosis, a non-customized orthosis may exert forces which may not be targeted and may be unnecessary.

Even targeted forces may cause a patient some discomfort which may challenge the patient's compliance and quality of wearing the orthosis. Therefore, it is important to monitor patient compliance and quality of wear.

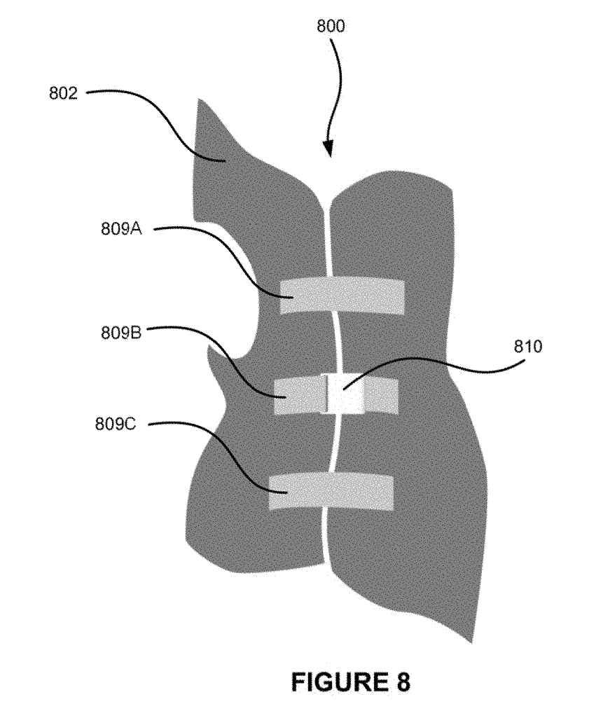

FIG. 8 illustrates an exemplary custom orthosis 800 and an orthosis monitoring device 110. Orthosis monitoring device 810 in FIG. 8 may be similar to the orthosis monitoring device 110 in FIG. 1. Custom orthosis 800 includes a housing 802, and straps 809A, 809B, and 809C. Although three straps are shown for exemplary purposes, more or less straps may be included. Although in the depicted implementation, the straps are placed in the front and center of housing 802, in another implementation, one or more of the straps may be placed elsewhere (e.g., on the side or the back of housing 802). Furthermore, although one orthosis monitoring device is shown as attached to one strap, in other implementations, additional monitoring devices may be included. In yet another implementation, an orthosis monitoring device may be capable of monitoring multiple straps.

Each strap 809A, 809B, and 809C may be associated with a relative value or an absolute value of strap pressure. The clinician, based on a patient's characteristics, may determine the strap pressure. The relative and absolute strap pressure values are correlated with optimum in-orthosis balancing which may be used to develop a set point for an orthosis monitoring device.

In an implementation, orthosis monitoring device 810 may be built into or otherwise removably attachable to a buckle that attaches to strap 809B. In some implementations, use of orthosis monitoring device 810 may encourage patients to comply with the prescribed amount of orthosis use and quality of wear of the orthosis. That is, if patients are aware that their compliance and quality of wear is tracked by the orthosis monitoring device, patients may be more motivated to wear the orthosis for the prescribed time period than if no tracking was provided.

Studies such as Bracing in Adolescent Idiopathic Scoliosis Trial (BrAIST) have demonstrated that the efficacy of orthosis is related to both in-orthosis correction and time spent using the orthosis. However, when the progress of adolescents who were prescribed pre-fabricated orthosis was tracked, the spinal deformities in a significant portion of adolescents still progressed. Some possible reasons for the progression of spinal deformities may be patients not complying with the orthosis and patients not wearing the orthosis in a correct manner.

In some implementations, orthosis monitoring device 810 may measure location, temperature, pressure, light, sound, movement and one or more of these variables over time. The orthosis monitoring device may include sensors such as a pressure sensor, a global positioning system, a clock or a thermometer. In an implementation, the pressure sensor may be a strain gauge that measures the pressure of a strap coupled to the orthosis monitoring device. Measurements or factors captured by the orthosis monitoring device include strap pressure (in terms of pounds per square inch), a location (in terms of coordinates), a temperature (measured in degrees Celsius or Fahrenheit), or a period of time (measured in seconds, minutes, etc.).

The thermometer may be used to ensure that a patient is wearing the orthosis. For example, if the temperature is a patient's body temperature, then the patient is deemed to be wearing the orthosis. If not, it is determined that the orthosis is not being worn.

In an implementation, factors including temperature or time period may be used by the orthosis monitoring device to determine compliance. In another implementation, factors including strap pressure or location may be used to determine orthosis quality of wear.

In an implementation, the global positioning system may be used to accurately determine a portion of the body (as it relates to the orthosis) to which to exert a force. In another implementation, the global positioning system may be used to determine whether the orthosis is properly aligned with the patient's body.

The orthosis monitoring device may measure multiple factors associated with the orthosis or patient or both. The orthosis monitoring device may provide an alert to a user when one of the factors (e.g., strap pressure) is not within an acceptable absolute or relative predetermined threshold (e.g., a predetermined strap pressure number or range). The acceptable pressure range may be predefined by a prescription for an orthosis and may be based on the pelvic incidence, sagittal balance, curve characteristic data, surface topography data and/or other factors associated with correction of a patient's spinal condition.

The orthosis monitoring device may track and measure compliance and quality of the orthosis. In an implementation, each strap may be associated with respective factors. In an implementation, a user wearing the orthosis and the orthosis monitoring device(s) may be alerted of an unacceptable strap tension range or issue with compliance by a visual indicator, audio indicator, and/or haptic indicator provided by the orthosis monitoring device. In an implementation, a visual indicator may provide illumination of one or more lights (e.g., light-emitting diodes (LEDs)). In an implementation, an audio indicator may provide a warning sound or phrase. In an implementation, a haptic indicator may provide a vibration.

The orthosis monitoring device may alert the patient or other users (i.e., the patient's family members, the patient's medical staff, etc.) by transmitting an alert to be displayed on one or more user devices. The orthosis monitoring device may monitor compliance and quality in real-time and provide alerts accordingly.

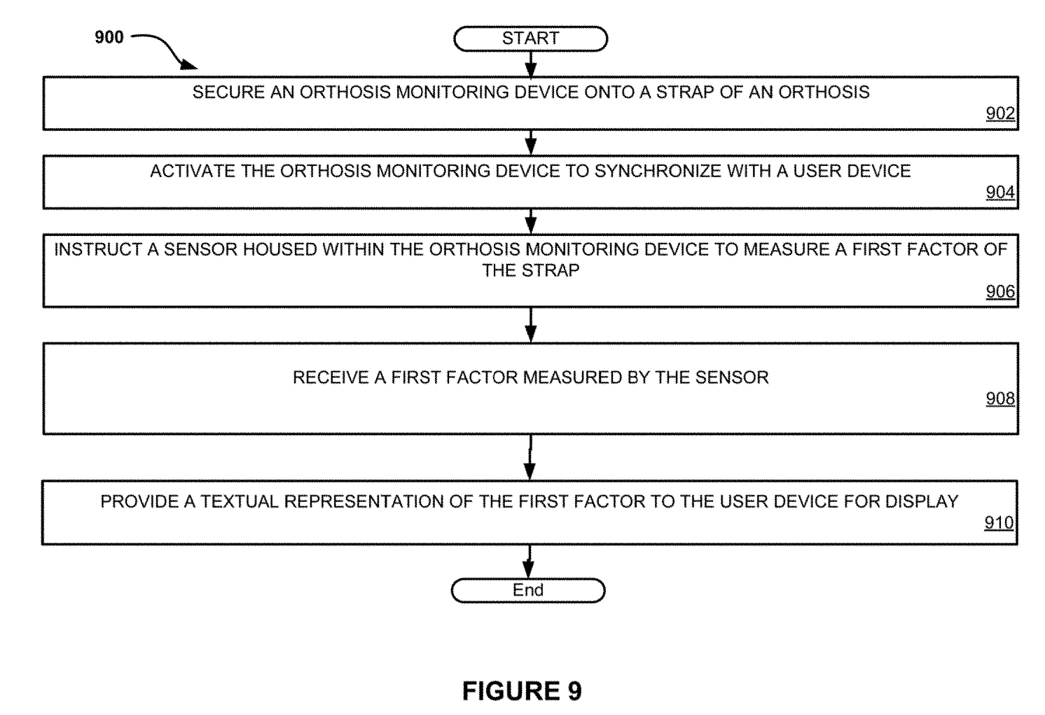

FIG. 9 is a flow chart illustrating a method 900 of implementing an orthosis monitoring device, according to an implementation of the disclosure. The steps of FIG. 9 may be a continuation of step 708 in FIG. 7. At step 902, an orthosis monitoring device is secured onto a strap of an orthosis. Referring now to FIG. 8, orthosis monitoring device 810 may be secured onto a strap 809B of custom-fabricated orthosis 800.

At step 904, the orthosis monitoring device is activated to synchronize with a user device. Referring to FIG. 1, orthosis monitoring device 110 may be activated (i.e., turned on) to synchronize with user device 116. The synchronization may involve connecting to the user device wirelessly or by other means. In an implementation, a secure login may be used to establish the synchronization. For example, a user may use communication interface 112 to enter a password, use biometric scanning or use other means of securely connecting to user device 116.

At step 906 of FIG. 9, a sensor housed within the orthosis monitoring device is instructed to measure a first factor of the strap. Referring now to FIG. 1, sensor(s) 120 housed within orthosis monitoring device 110 is instructed to measure the first factor.

At step 908 of FIG. 9, a first factor measured by the sensor is received. As described above, the first factor may include strap pressure, location, temperature, pressure, light, movement and/or sound including the change(s) of these values over a time period.

At step 910, a textual representation of the first factor is provided to the user device for display. Referring again to FIG. 1, orthosis monitoring device 110 transmits the first factor to user device 116, via network 114 or by other means.

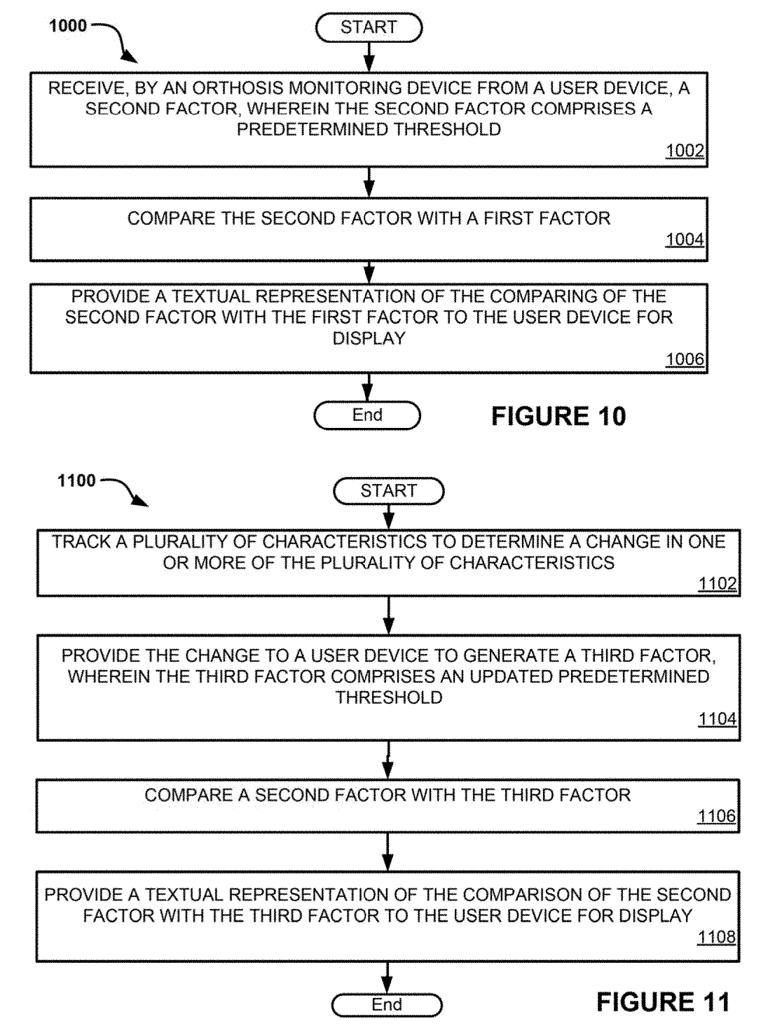

FIG. 10 is a flow chart 1000 illustrating a method for comparing factors at an orthosis monitoring device. The steps of FIG. 10 may be a continuation of step 910 in FIG. 9. At step 1002, a second factor is received by an orthosis monitoring device from a user device. The second factor includes a predetermined threshold. Referring now to FIG. 1, orthosis monitoring device 110 receives a second factor, which includes a predetermined threshold (i.e., in an absolute or relative form), from user device 116.

At step 1004 of FIG. 10, the second factor is compared with the first factor. The orthosis monitoring device compares the two factors. For example, suppose that the user device sets a relative predetermined threshold for a strap pressure of thirty pounds per square inch (PSI) to thirty-seven PSI. The measured factor may be thirty-five PSI. Therefore, when performing the comparison, the orthosis monitoring device determines that the measured factor is within range of the predetermined threshold. In another implementation, the predetermined threshold may be an absolute threshold (i.e., an exact number).

At step 1006 of FIG. 10, a textual representation of the comparing of the second factor with the first factor is provided to the user device for display. The user device may show the results of the comparison to the clinician, patient, and/or patient's designees.

FIG. 11 is a flow chart 1100 illustrating a method for tracking characteristics to determine a change. The steps of FIG. 11 may be a continuation of step 1006 in FIG. 10. At step 1102, multiple characteristics are tracked to determine a change in the multiple characteristics. For example, the clinician may access a patient to determine a change in a characteristic such as pelvic incidence (which effects sagittal balance). The clinician may determine that a change to a factor is to be made. For example, the strap pressure, the time the patient is to use the orthosis or the location of the orthosis may change. The new factor is updated in view of the change.

At step 1104, the change is provided to a user device to generate a third factor. The third factor includes an updated predetermined threshold.

At step 1106, a second factor is compared with the third factor.

At step 1108, a textual representation of the comparison of the second factor with the third factor is provided to the user device for display.

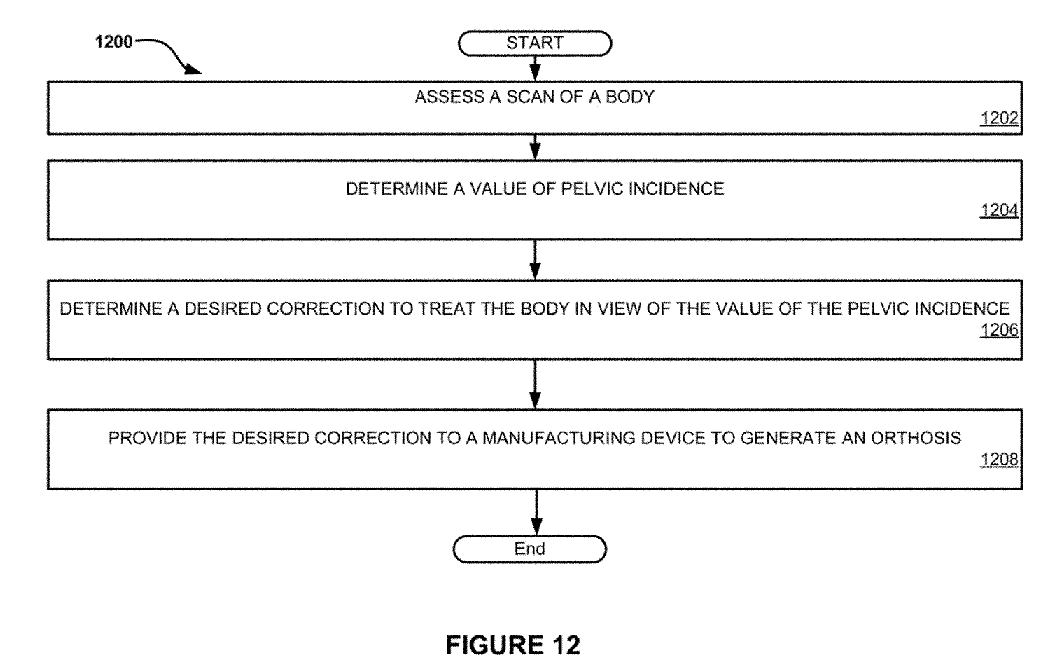

FIG. 12 is a flowchart 1200 illustrating a method for prescribing a customized orthosis based on assessment of a scan of a body. At step 1202, a scan of a body is assessed. Referring to FIG. 1, a clinician scans a body of a patient to assess and prescribe a correction. The medical personnel assessment and prescription for correction 124 may be based on performing a scan of the body (either partially or as a whole) captured by an x-ray, an EOS scan and/or a magnetic resonance imaging.

At step 1204 of FIG. 12, a value of pelvic incidence is determined from the scan. As shown in FIG. 1, the pelvic incidence is stored and accessible via patient characteristics 122.

At step 1206 of FIG. 12, a desired correction to treat the body in view of the pelvic incidence and sagittal balance is determined. The desired correction is included in medical personnel assessment and prescription for correction 124 in FIG. 1.

At step 1208 of FIG. 12, the desired correction is provided to a manufacturing device to generate an orthosis.

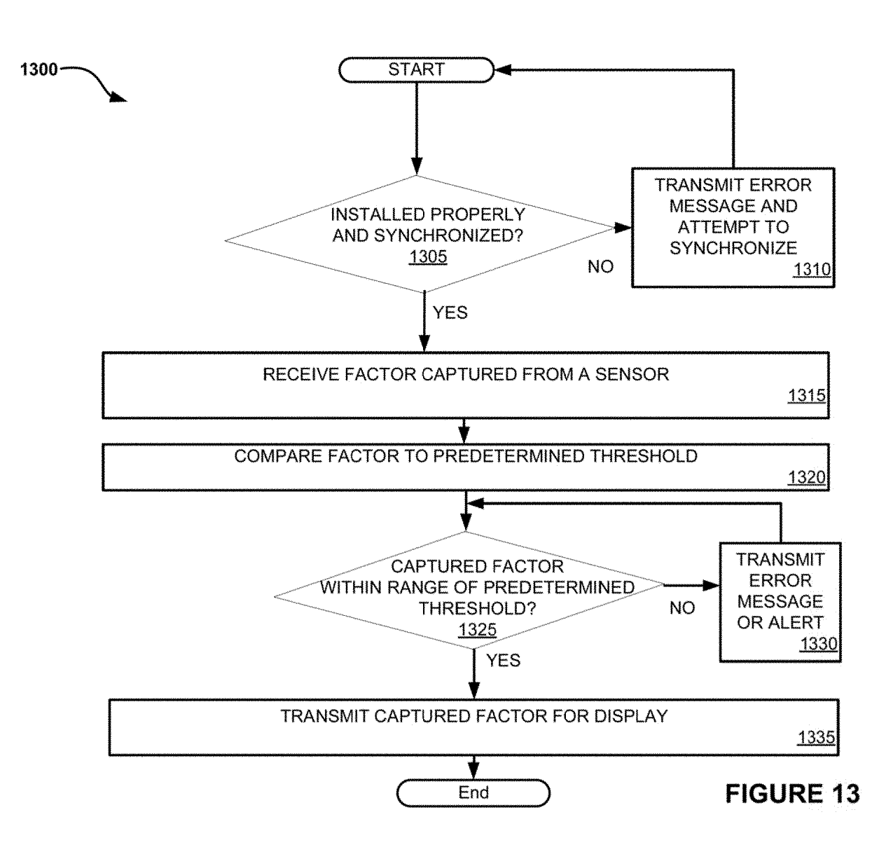

FIG. 13 is a flowchart 1300 illustrating a method for comparing factors for providing a message or alert. The steps of FIG. 13 may be a continuation of step 1208 in FIG. 12. At decision block 1305, it is determined whether the orthosis monitoring device is installed properly and synchronized with a user device. If the decision block yields a “no”, at block 1310, an error message is transmitted to the user device and/or the orthosis monitoring device, and an attempt at resynchronization is made. The method then returns to decision block 1305.

If decision block 1305 yields a “yes”, the method continues to block 1315, and a factor is captured from a sensor.

At block 1320, the factor is compared to a predetermined threshold.

At decision block 1325, it is determined whether the captured factor is within a range of the predetermined threshold. If the decision block yields a “no”, at block 1330, an error message is transmitted to the user device and/or an alert is transmitted to the orthosis monitoring device. The method returns to decision block 1325.

If decision block 1325 yields a “yes”, the method continues to block 1335 and the captured factor is transmitted for display on the user device.

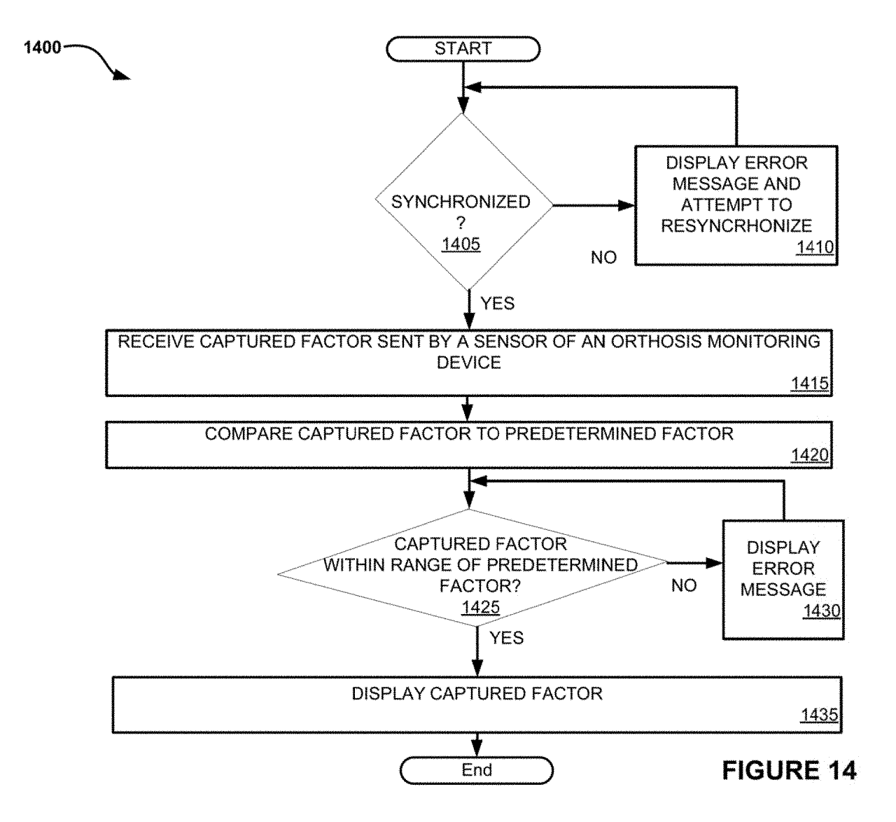

FIG. 14 a flowchart 1400 illustrating a method for providing a display at a user device. At decision block 1405, it is determined whether the user device is synchronized. If the decision block yields a “no”, at block 1410, an error message is displayed on the user device and an attempt is made to resynchronize. The method returns to decision block 1405.

If decision block 1405 yields a “yes”, the method continues to block 1415, and a captured factor sent by a sensor of an orthosis monitoring device is received.

At block 1420, the captured factor is compared to a predetermined factor.

At decision block 1425, it is determined whether a captured factor is within a range of a predetermined factor. If the decision block yields a “no”, the method continues to block 1430 and an error message is displayed.

If decision block 1425 yields a “yes”, the method continues to block 1435 and a captured factor is displayed to a user.

Thus the orthosis monitoring device tracks and ensures not only compliance but also ensures proper fit and wear which correlate with optimum in-orthosis correction and balance.

In an implementation, the orthosis monitoring device may communicate with one or more user devices using wireless communications such as Bluetooth. Feedback with respect to patient compliance and quality of wear of an orthosis may be provided to the user devices (used by families, caretakers, clinicians, etc.) using an application such as a mobile application or app.

In an implementation, by actively monitoring compliance and quality of an orthosis, patients may be encouraged to properly use the orthosis as described. For example, if a child uses a prescribed orthosis for a prescribed period of time and uses the straps having a correct strap pressure, the orthosis monitoring device may report the compliance and quality to the child's parent and physician. The child's parent may then reward the child based on appropriate use of the orthosis. Thus, the child may be encouraged to use the orthosis as prescribed.

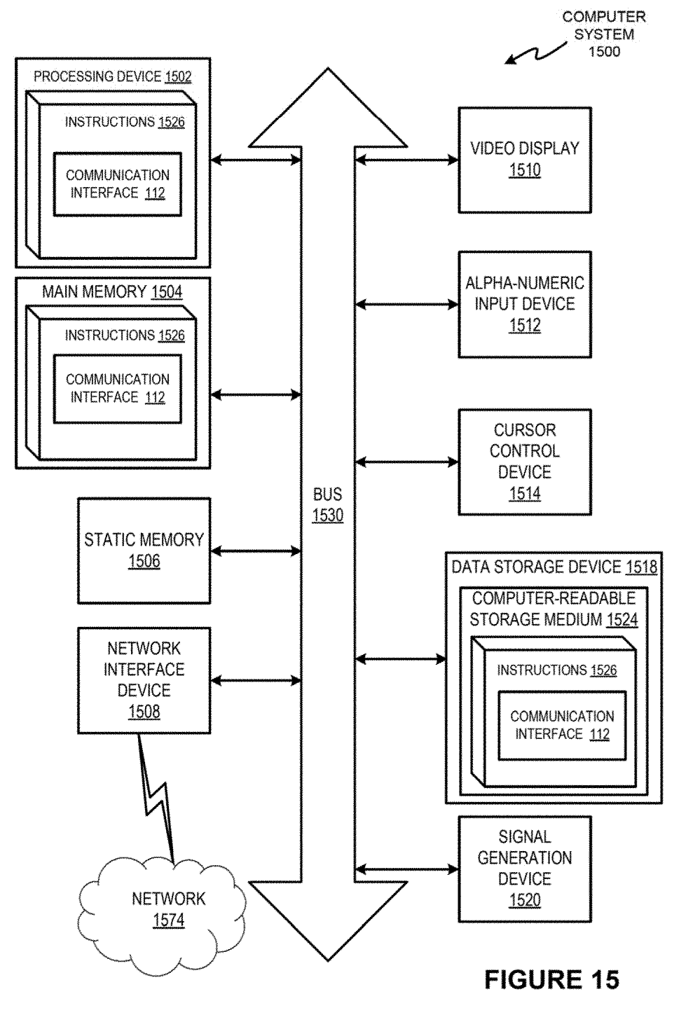

FIG. 15 illustrates a block diagram of an illustrative computing device operating in accordance with the examples of the disclosure. In alternative implementations, the machine may be connected (e.g., networked) to other machines in a LAN, an intranet, an extranet, or the Internet. The machine may operate in the capacity of a server or a client device in a client-server network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The computer system 1500 includes a processing device 1502, a main memory 1504 (e.g., read-only memory (ROM), flash memory, dynamic random access memory (DRAM) (such as synchronous DRAM (SDRAM) or DRAM (RDRAM), etc.), a static memory 1506 (e.g., flash memory, static random access memory (SRAM), etc.), and a data storage device 1518, which communicate with each other via a bus 1530.

Processing device 1502 represents one or more general-purpose processing devices such as a microprocessor, central processing unit, or the like. More particularly, the processing device may be a complex instruction set computing (CISC) microprocessor, reduced instruction set computer (RISC) microprocessor, very long instruction word (VLIW) microprocessor, or processor implementing other instruction sets, or processors implementing a combination of instruction sets. Processing device 1502 may also be one or more special-purpose processing devices such as an application specific integrated circuit (ASIC), a field programmable gate array (FPGA), a digital signal processor (DSP), network processor, or the like. The processing device 1502 is configured to execute the instructions 1526 for performing the operations and steps discussed herein.

In accordance with one or more aspects of the present disclosure, processing device 1502 may be configured to execute communication interface 112 implementing a portion or all of methods in flowcharts 1000, 1100, 1200 and 1300 for communicating information based on monitoring a patient wearing an orthosis.

The computer system 1500 may further include a network interface device 1508 communicably coupled to a network 1574. The computer system 1500 also may include a video display unit 1510 (e.g., a liquid crystal display (LCD) or a cathode ray tube (CRT)), an alphanumeric input device 1512 (e.g., a keyboard), a cursor control device 1514 (e.g., a mouse), and a signal generation device 1520 (e.g., a speaker).

The data storage device 1518 may include a computer-readable storage medium 1524 on which is stored instructions 1526 embodying any one or more of the methodologies of functions described herein. The instructions 1526 may also reside, completely or at least partially, within the main memory 1504 as instructions 1526 and/or within the processing device 1502 as instructions 1526 during execution thereof by the computer system 1500; the main memory 1504 and the processing device 1502 also constituting machine-accessible storage media.

In accordance with one or more aspects of the present disclosure, instructions 1526 may comprise executable instructions encoding various functions of communication interface 112 implementing a portion or all of methods in flowcharts 1000, 1100, 1200 and 1300 for communicating information based on monitoring a patient wearing an orthosis.

While the computer-readable storage medium 1524 is shown in an example implementation to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any media that is capable of storing, encoding or carrying a set of instruction for execution by the machine and that cause the machine to perform any one or more of the methodologies of the disclosure. The term “computer-readable storage medium” shall accordingly be taken to include, but not be limited to, solid-state memories, and optical and magnetic media.

In the foregoing description, numerous details are set forth. It may be apparent, however, that the disclosure may be practiced without these specific details. In some instances, well-known structures and devices are shown in block diagram form, rather than in detail, in order to avoid obscuring the disclosure.

Some portions of the detailed descriptions are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, as apparent from the following discussion, it is appreciated that throughout the description, discussions utilizing terms such as “receiving”, “initiating”, “generating”, “determining”, “sending”, “invoking”, “storing”, “updating”, “identifying”, “presenting”, “causing”, or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The disclosure also relates to an apparatus for performing the operations herein. This apparatus may be specially constructed for the required purposes, or it may comprise a general purpose computer selectively activated or reconfigured by a computer program stored in the computer. Such a computer program may be stored in a machine readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

The algorithms and displays presented herein are not inherently related to any particular computer or other apparatus. Various general purpose systems may be used with programs in accordance with the teachings herein, or it may prove convenient to construct a more specialized apparatus to perform the required method steps. The required structure for a variety of these systems may appear as set forth in the description below.

The disclosure may be provided as a computer program product, or software, that may include a machine-readable storage medium having stored thereon instructions, which may be used to program a computer system (or other electronic devices) to perform a process according to the disclosure. A machine-readable storage medium includes any mechanism for storing or transmitting information in a form readable by a machine (e.g., a computer). For example, a machine-readable (e.g., computer-readable) medium includes a machine (e.g., a computer) readable storage medium (e.g., read only memory (“ROM”), random access memory (“RAM”), magnetic disk storage media, optical storage media, flash memory devices, etc.), etc.

Whereas many alterations and modifications of the disclosure may no doubt become apparent to a person of ordinary skill in the art after having read the foregoing description, it is to be understood that any particular example shown and described by way of illustration is in no way intended to be considered limiting. Therefore, references to details of various examples are not intended to limit the scope of the claims.

It will be understood that the implementations described herein are merely exemplary and that a person skilled in the art may make many variations and modifications without departing from the spirit and scope of the invention. All such variations and modifications are intended to be included within the scope of the invention.

What is claimed is:

1. A method comprising:
performing a scan of a body;
determining one of a plurality of characteristics associated with the body in view of the scan, the one of the plurality of characteristics comprising sagittal balance;
determining desired correction of the body in view of the sagittal balance; and
providing the desired correction to a manufacturing device to generate an orthosis, wherein the orthosis comprises a housing and a strap generated in view of the desired correction.

2. The method of claim 1, wherein the plurality of characteristics further comprises a pelvic incidence and the pelvic incidence is calculated as a sum of a value of a sacral slope and a value of a pelvic tilt, wherein the value of the sacral slope and the value of the pelvic tilt are determined by analyzing the scan.

3. The method of claim 1, wherein the performing the scan of the body comprises imaging the body by performing at least one of an x-ray, an EOS scan and magnetic resonance imaging.

4. The method of claim 3 wherein the plurality of characteristics further comprises at least one of curve characteristics data and surface topography data extracted from at least one of the x-ray, the EOS scan and the magnetic resonance imaging.

5. The method of claim 1, further comprising:
tracking the plurality of characteristics to determine a change in one or more of the plurality of characteristics;
updating the desired correction to generate an updated desired correction in view of the change; and
providing the updated desired correction to the manufacturing device to generate an updated orthosis.

6. The method of claim 1, further comprising:
securing an orthosis monitoring device onto the strap of the orthosis;
activating the orthosis monitoring device to synchronize with a user device;
instructing a sensor housed within the orthosis monitoring device to measure a first factor of the strap;
receiving the first factor measured by the sensor; and
providing a textual representation of the first factor to the user device for display.

7. The method of claim 6 wherein the first factor comprises one of a strap pressure, a location, a temperature or a time period and wherein the sensor comprises one of a strain gauge, a global positioning system or a thermometer.

8. The method of claim 6 wherein the desired correction comprises one of a correction of a frontal balance of the body within a frontal balance correction range or correction of the sagittal balance within a sagittal balance correction range.

9. The method of claim 6, further comprising:
receiving, by the orthosis monitoring device from the user device, a second factor, wherein the second factor comprises a predetermined threshold;
comparing the second factor with the first factor; and
providing a textual representation of the comparing of the second factor with the first factor to the user device for display.

10. The method of claim 9, further comprising:
tracking the plurality of characteristics to determine a change in one or more of the plurality of characteristics;
providing the change to the user device to generate a third factor, wherein the third factor comprises an updated predetermined threshold;
comparing the second factor with the third factor; and
providing a textual representation of the comparing of the second factor with the third factor to the user device for display.

11. A method comprising:
assessing a scan of a body;
determining a value of pelvic incidence from the scan;
determining a desired correction to treat the body in view of the value of pelvic incidence; and
providing the desired correction to a manufacturing device to generate an orthosis.

12. The method of claim 11 wherein the value of pelvic incidence is calculated as a sum of a value of a sacral slope and a value of a pelvic tilt, wherein the value of the sacral slope and the value of the pelvic tilting are determined by the assessing the scan.

13. The method of claim 11 wherein the orthosis comprises a housing and a strap generated in view of the desired correction.

14. The method of claim 13, further comprising:
securing an orthosis monitoring device onto the strap of the orthosis;
activating the orthosis monitoring device to synchronize with a user device;
instructing a sensor housed within the orthosis monitoring device to measure a first factor of the strap;
receiving the first factor measured by the sensor; and
providing a textual representation of the first factor to the user device for display.

15. The method of claim 14, further comprising:
receiving, by the orthosis monitoring device from the user device, a second factor, wherein the second factor comprises a predetermined threshold, wherein the predetermined threshold comprises one of an absolute or relative value of one of a strap pressure, a location, a temperature or a time period.

16. The method of claim 15, further comprising:
determining whether the first factor is within the predetermined threshold of the second factor;
in response to determining that the first factor is not within a range of the predetermined threshold, providing, to the user device for display, an error message indicative of a difference between the first factor and the predetermined threshold; and
in response to determining that the first factor is within the range of the predetermined threshold, providing, to the user device for display, a textual representation of the first factor.

17. The method of claim 15, further comprising:
determining whether the first factor is within the predetermined threshold of the second factor; and
in response to determining that the first factor is not within the range of the predetermined threshold, transmitting an alert, wherein the alert comprises at least one of a visual indicator, audio indicator, and haptic indicator.

18. An apparatus comprising:
a database configured to store an input of sagittal balance associated with a scoliotic spine, wherein input of the sagittal balance comprises an input of a value of pelvic incidence associated with the scoliotic spine and
a device configured to store a prescription, wherein the prescription comprises a desired correction of the scoliotic spine determined in view of the sagittal balance;
wherein a custom orthosis is manufactured by a manufacturing device in view of the desired correction.

19. The apparatus of claim 18, further comprising an orthosis monitoring device coupled to the custom orthosis, the orthosis monitoring device configured to measure a factor associated with the custom orthosis.

20. The apparatus of claim 18, further comprising a sensor housed within the orthosis monitoring device to measure the factor.
